(19) United States
(12) Patent Application Publication
(10) Pub. No.: US 2005/0098179 A1
(43) Pub. Date: May 12, 2005

(54) Multi-level positive air pressure method and delivery apparatus

(76) Inventors: Steve Burton, Midlothian, VA (US); Paul Boatner, Midlotian, VA (US)

Correspondence Address: JOHN H. THOMAS, P.C., 1561 EAST MAIN STREET, RICHMOND, VA, 23219, US

(21) Appl. No.: 10/703,219

(22) Filed: Nov. 6, 2003

(51) Int. Cl. A61M 16/00

(52) U.S. Cl. 128/205.24; 128/204.18; 128/205.25; 128/204.23

(57) ABSTRACT

A method and apparatus provides multi-level positive air pressure to the airway of a patient for breathing by the patient. An air flow generator is connected to a plurality of valves and hoses connected to and adapted to control and direct the flow of air from the air flow generator. A mask is connected to the air flow generator by the hoses, the mask adapted to deliver air to the airway of a patient. The plurality of valves deliver multiple, different levels of positive airway pressure to the airway of a patient. Alternatively, the air flow generator may include a plurality of blower apparatuses connected to the corresponding plurality of valves and hoses.

This invention relates to a method and related air delivery apparatus for delivering multiple levels of positive air pressure to the airway of a patient.

### BACKGROUND OF THE INVENTION

Sleep apnea is a condition suffered by a significant portion of the general population. In simple terms, sleep apnea results from temporary obstruction of a person's upper airway during sleep. Persons having sleep apnea experience a broad range of physical symptoms. In mild cases, the symptoms may simply reveal themselves as tiredness or sleepiness during the day. In more serious cases of sleep apnea, the symptoms can be severe and debilitating.

Details of the physical mechanism of the upper airway obstruction in sleep apnea patients have been widely studied. Although many physical conditions or abnormalities have been identified as potential causes for different sleep apnea cases, it is simply known that the upper airway may narrow or close during the sleep of patients suffering from sleep apnea. Regardless of the cause, the airway obstruction results in increased air flow resistance during inhalation of the patients suffering from sleep apnea. Both medical and surgical options have been used and explored to treat sleep apnea. Obviously, many of these treatments constitute substantial undertakings with some associated risks.

One area of treatment of sleep apnea includes the use of continuous positive airway pressure (CPAP) to maintain the airway of the patient in a constantly open state during sleep. An example and discussion of sleep apnea treatments based on continuous positive airway pressure is set forth in U.S. Pat. No. 4,655,213. A related treatment involves the use of bi-level positive airway pressure to maintain the open state of the airway of a patient during sleep. U.S. Pat. No. 5,148,802 is an example of such a bi-level treatment and related apparatus.

Although both continuous and bi-level positive airway pressure have been determined to be very effective and acceptable to many patients, those treatments do have some drawbacks, at least to a segment of sleep apnea sufferers. A substantial number of sleep apnea patients do not like or tolerate continuous and bi-level positive airway pressure well. Research studies suggest that as many as one half to one third of the people using CPAP and bi-level therapy stop using the therapy within one year; treatment side-effects are the most common reason sited for discontinuing its use.

Also, while breathing mask products commercially broadly used with positive airway pressure systems have no or insignificant moisture build up problems, future products may have intermittent air flow characteristics. A potential problem in reducing or spacing out airflow through a mask is that condensation can build up in the mask. This condensation buildup could cause discomfort.

### SUMMARY OF THE INVENTION

Accordingly it is the object of the present invention to overcome the foregoing drawbacks and to provide a method and air delivery apparatus for creating multiple levels of positive air pressure within the airway of a patient.

In one embodiment, a method of providing multi-level positive air pressure to the airway of a patient for breathing by the patient includes providing an air flow generator and a plurality of valves and hoses connected to and adapted to control and direct the flow of air from the air flow generator. The method also includes providing a mask that is connected to the air flow generator by the hoses, the mask adapted to deliver air to the airway of a patient. The method finally includes actuating the plurality of valves to deliver multiple, different levels of positive air pressure to the airway of a patient. The air flow generator may comprise a plurality of blower apparatuses with the plurality of valves and hoses connected to the corresponding plurality of blower apparatuses. The plurality of blower apparatuses may each deliver different air flows. The method may further include providing one or a plurality of physiological detectors adapted to detect biological triggering events, wherein the detector or detectors are connected to and actuate the valves. The physiological detectors may identify a plurality of different biological triggering events. One of the positive air pressures may constitute a bias flow of air to the mask.

Alternatively, the invention includes an apparatus for providing multi-level positive air pressure to the airway of the patient for breathing by the patient. The apparatus comprises an air flow generator and a plurality of hoses connected to the air flow generator and adapted to direct the air from the air flow generator. The apparatus further comprises a corresponding plurality of valves connected to the plurality of hoses and adapted to control the flow of air from the air flow generator. A mask is connected to the air flow generator by the hoses, the mask being adapted to deliver air to the airway of a patient. Alternatively, the air flow generator may comprise a plurality of blower apparatuses, and the plurality of valves and hoses are connected to the corresponding plurality of blower apparatuses. The plurality of blower apparatuses may each deliver different air flows. The apparatus may further comprise a physiological detector or plurality of physiological detectors adapted to detect biological triggering events, and wherein each detector is connected to and actuates the valves.

Also, the invention includes a method of providing multi-level positive air pressure to the airway of a patient for breathing by a patient. The method comprises providing an air flow generator, wherein the generator can provide multiple, different air flows. The method also includes providing a hose and a mask, the hose connected to the air flow generator and the mask to deliver air from the generator to the mask. The mask is adapted to deliver the air to the airway of a patient. The method further includes actuating the air flow generator to deliver multiple, different levels of positive air pressure to the air way of a patient. One of the positive air pressures may constitute a bias flow of air to the mask. The method may further include providing a physiological detector adapted to detect a biological triggering event, wherein the detector is connected to and actuates the air flow generator.

Additionally, the invention includes a method of providing multi-level positive air pressure to the airway of a patient for breathing by the patient. The method includes providing an air flow generator and a valve and hose connected to and adapted to control and direct the flow of air from the air flow generator. The valve is adapted to allow multiple, different levels of air to flow through it. The method includes providing a mask that is connected to the air flow generator by the hose, the mask adapted to deliver air to the airway of a patient. The method finally includes actuating the valve to deliver multiple, different levels of positive air pressure to the airway of a patient. One of the levels of positive air pressure may constitute a bias flow of air to the mask. The method may alternatively further include providing a physiological detector adapted to detect a biological triggering event, wherein the detector is connected to and actuates the valve.

Still further alternatively, the invention includes a breathing mask for use in connection with a positive air flow source. The mask includes a flexible interface adapted to fit around the nose and/or mouth of a patient. The mask further includes a rigid retainer adapted to fit securely around the flexible interface. The rigid retainer may be adapted to be removably fitted around the flexible interface.

### BRIEF DESCRIPTION OF THE DRAWINGS

FIG. 1 is a schematic flow diagram demonstrating the present invention.

FIG. 2 is a diagram of an apparatus in accordance with the present invention.

FIGS. 3-7 are schematic diagrams of alternative valve and air flow systems.

FIGS. 8A and 8B display breathing mask constructions in accordance with the present invention.

### DETAILED DESCRIPTION OF THE INVENTION

The present invention relates to a method of and related apparatus for providing multiple levels of positive air pressure to the airway of a patient suffering from sleep apnea or related air patency disorder.

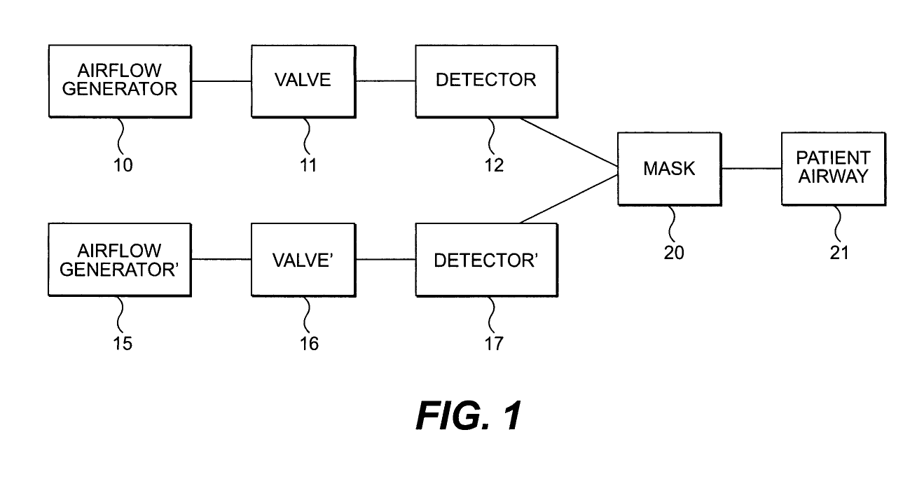

FIG. 1 is a schematic flow chart illustrating the conceptual operation of a preferred embodiment of the present invention. FIG. 1 illustrates two air flow generators 10 and 15 that each provide a flow of air at positive air pressure to a mask 20 in order to maintain the patency of a patient's airway 21. Each air flow generator 10 and 15 includes a valve 11 and 16 and detector 12 and 17 respectively. The valves 11 and 16 open and close to allow the staggered or combined flow of air into the mask 20. The detectors 12 and 17 operate themselves or through a further processing actuator (not shown) to actuate the valves 11 and 16 to open and shut. Therefore, the air flow into the mask 20 is regulated and controlled in accordance with parameters predetermined and input into the detectors 12 and 17. Of course, three or more air flow generators may be used—more than simply the two demonstrated in this schematic.

Even in the simple schematic demonstrated in FIG. 1, up to four levels of airway pressure may be delivered to a mask such as mask 20. The first and lowest level of airway pressure is simple ambient. This would be the situation where both valves 11 and 16 are closed. Second and third levels of airway pressure may be delivered when one of the valves 11 or 16 is opened and the other is closed. The second and third levels of airway pressure assume that the air flow generators 10 and 15 deliver different air flows. The fourth level of airway pressure demonstrated in FIG. 1 would occur when both valves 11 and 16 are open such that the volume of air delivered to the mask 20 is the combined volume of air flow from the air flow generators 10 and 15. Accordingly, even the simple schematic shown in FIG. 1 demonstrates a quad-level of positive airway pressure. As other air flow generators could be added to the system, it is possible to imagine the exponential variations in the amount of air flow that could be delivered to a mask such as mask 20.

In one preferred embodiment, one of the flows of air that may be directed to a mask is a low level, sub-therapeutic amount of air referred to herein as a bias flow of air. Inherently, if the bias flow is combined with other therapeutic volumes of air delivered concurrently therewith, then part of that therapeutic delivery includes the bias flow. But the bias flow alone may serve the purpose of ventilation and reducing condensation within the mask to be worn by the patient.

In a still further embodiment, an air flow generator such as air flow generator 10 may be engineered or specified to itself provide multiple different air flows. That is, the mechanism/blower within the generator itself may be variably actuated to provide different rates of flow of air from the generator. The different rates of air flow, preferably three or more, allow a system to deliver multiple, different levels of positive air pressure to the airway of a patient. In this alternative, only a single air flow generator would be necessary to create the multiple levels of air flow to a mask and, thereafter, a patient's airway.

In a still further embodiment, a single valve such as valve 11 may be adapted to allow multiple, different levels of air to flow through it. In this way, a single air flow generator connected to such a valve can, through the different actuation levels of the valve, deliver multiple, different levels of positive air pressure to a mask. These variable speed air flow generators or air flow valves may, as noted herein, also be combined to create further multiples of different positive air pressure available to be delivered to a patient's airway.

The following discusses an apparatus and method for the treatment of apnea and related air patency disorders as it is presently being developed. Those of skill in the art will be able to design and develop treatments and apparatuses that are functionally the same or perform the same functions as those noted herein. The present invention is not limited to the specific structure of this detailed description.

The air flow generators 10 and 15 may be any type of commercially available blower or other compressor. Typically, the range of requirements for air flow by such a generator includes the ability to deliver about 3 centimeters of H₂O as a minimum flow rate up to about 18 centimeters of H₂O. An air flow generator for use in accordance with the present invention could possibly be the same as that used in connection with conventional continuous positive airway pressure and bi-level positive airway pressure equipment. Commercially available products include blowers manufactured by Respironics, ResMed and Viasys Healthcare.

The air flow generators 10 and 15 are preferably separate devices. However, it can be engineered that the air flow generators may be the actual same device but have different-sized orifices or tubes that divert the air from the device and that thereby create different air flows. In this way, there could functionally be different air flow generators that provide the different flows of air to a mask even though there is only one physical blower. Also, as noted earlier, a single air flow generator could itself have multiple, different air flow capabilities. At present, it is believed that different mechanical devices are preferred for purposes of servicing and better controlling the different air flow amounts. However, lower cost of manufacturing may favor the use of a single blower.

Air tubes connect the air flow generators 10 and 15 to the mask 20. These air tubes are intended to be of a conventional structure. For instance, an air tube may be a 25 mm bore hose. As noted earlier, in the event that the same air flow generator is used to create the different flows of air, then multiple air tubes may have a different diameter in order to create those different air flows. Preferably, the multiple air tubes will be braided or otherwise attached to each other to minimize tangling or other limitations on operation. This merely simplifies the overall structure to be used by a patient.

A mask that is envisioned for use in connection with the present invention may be any type of mask commercially available and known. The mask may be an oral and/or nasal device that delivers air to the upper respiratory system. A cannula or structure that delivers the air directly into the nares, sometimes referred to as a nasal pillow, may also be used. It is only necessary that, like some masks used in connected with current therapies, the mask have a valve to allow the escape of exhaled air by the patient.

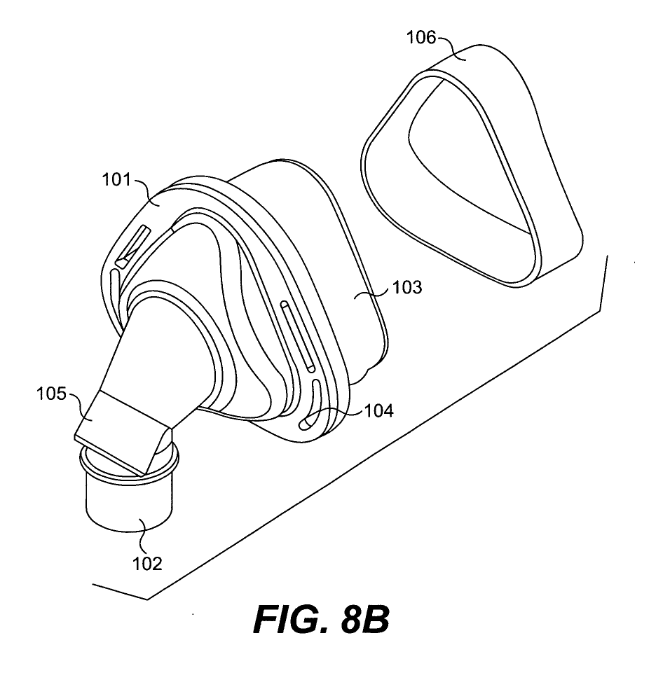

FIGS. 8A and 8B illustrate one preferred embodiment of a mask 100 that could be used in connection with the present invention. The mask 100 is made of a hard plastic shell 101 adapted to cover a patient's mouth and nose. The mask 100 also has a hose connection aperture 102 and slots 104 adapted to receive head gear straps (not shown) for wearing by a patient. The mask 100 further includes a gel 103 which is a soft flap interface adapted to press against and seal against the face of a patient. The gel 103 is intentionally soft and pliable in order to be as comfortable as possible for the patient. Also, a check valve 105 is used in the mask 100. The check valve 105 provides a fail safe in the event that the therapy delivery system fails and the user needs access to fresh air directly. In the design discussed herein, a check valve is also required in a mask or nasal pillow that covers the nose only. In that the inhalation cycle may by design be only partially supported by the therapeutic delivery of air, the check valve in this design serves an additional feature of allowing the user to more easily gain air in order to complete their respiratory inhale.

Preferably, the mask 100 is a rigid construction and includes a shell overlay 106. The mask overlay 106 or retainer is rigid and is adapted to overlay the soft gel 103 flexible interface of the mask 100. Because the other components of the mask 100 are rigid, the entire construction (with the overlay 106) does not expand or contract substantially during the delivery of air to the patient. Most nasal masks currently available either have a gel or a soft, flexible interface (like the gel 106) where the mask fits around the nose. This flexible area causes problems with delivery of a specific puff of air because of the constant expanding and contracting of the mask with cycling of the puff. To alleviate this problem, a hard shell like overlay retainer 106 can be developed to fit securely around the flexible area of the mask still allowing the flexible material to conform to the nose, while at the same time containing the sidewalls to prevent expansion caused by the flow of air. If the construction is too flexible, then the different levels of airway pressure may not be effectively delivered to the patient as those different levels would be absorbed or damped by the mask expansion and contraction. Another alternative embodiment is a mask having a relatively minimal surface area so that it will only grasp the nose. In this way, there is not a substantial surface area on which moisture may be collected. An alternative preferred type of mask is one that is built-up on the face of a patient with a material that is relatively rigid to maintain its shape and that is custom fit for each patient.

The detectors 12 and 17 may be any type of physiological detector for identifying a triggering biological event in the patient. The detectors 12 and 15 may detect biological events such as snoring, apnea, or changes in upper airway resistence. The detectors 12 and 17 may detect different triggering events that could be ameliorated by the different air flows. A conventional physiological detector is one that identifies and picks up a patient's breathing cycle through pressure changes in the apparatus system. As the patient exhales, the pressure in the system increases. Inversely, as the patient inhales, the pressure in the system decreases. In this way, the detector may pick up all of the phases of the breathing cycle. It is preferred that a breathing cycle detector used in connection with the present invention be able to detect at least approximately −5 to +5 centimeters of H₂O. The actual detection range may be broader than this range. An alternative detector could be a flow rate detector capable of detecting a flow rate of from zero to one half liter of air.

Valves 11 and 16 control the flow of air from the blower 10 and 15 to the mask 20. Conventionally, the valves are placed inside the blower housing between the blower and a pressure transducer (physiological detector 12). The valve may be a customized valve to work closely for a given specification, or alternatively, an off the shelf valve able to open and shut the flow of air to the mask. The valves 11 and 16 are actuated by the physiological detectors 12 and 17. In an exemplary case of a pressure transducer that detects a patient's breathing cycle, a valve shuttles a spool from position A to position B via a solenoid. Alternative constructions include a ball valve or a knife gate type of valve.

The valves 11 and 16 may be actuated directly as a result of signals received from the detectors 12 and 17. In this case, a processor could be integrated into the detector or the valves to evaluate and act on the signals. Alternatively, there may be a separate actuator processor that receives signals from the detectors 12 and 17, processes them, then operates to drive the valves open or closed. A still further alternative construction would be one where there are no detectors such as 12 and 17. Instead, an actuator processor determines an automatic set of air flow parameters. There may be a manual setting of the air flow parameters. Still further alternatively, the actuator processor may receive signals from detectors such as detectors 12 and 17 and then operate off of those signals in a predetermined manner. This predetermined manner may be an automatic setting or it may be manual setting of air flow parameters. The various flow parameters could include the following. The actuation of the valves could be triggered off of a simple timer which merely drives the valves open and shut every x number of seconds. This simple timer alternative would operate regardless of the breathing cycle of the patient. Alternatively, the air flow may be actuated with the opening of the valves in coordination with the inhalation and exhalation of the breathing cycle of a patient. Finally the interval could be engineered to be up to as much as a continuous air flow from the plurality of air flow generators.

The amount and timing of the air flow in accordance with this multi-level positive air way pressure invention is intended to vary depending on the requirements of a given application. The minimum amount of flow from one or more of the air flow generators could be any amount above zero but preferably the minimum amount is in the range of 1-3 cm of water. A maximum flow rate for any one or more of the air flow generators would be approximately 8-10 cm of water. The purposes for the air flow are both therapeutic and, as noted earlier, for ventilation purposes to prevent excessive moisture build up. The therapeutic volume of flow could be any amount depending on the requirements and physical traits of the patient. The ventilation flow could be anywhere from as little as just above zero to up to 8-18 cm of water. The necessary ventilation flow amount will vary in accordance with ambient humidity, size of the patient, etc.

It is believed that one of the reasons that patients discontinue current CPAP and bi-level therapy is that the delivered air flow deviates from the normal patient experience during respiration. It is believed that the current invention of delivery multiple levels of positive air pressure flow, preferably three or more different levels (rates) of air flow may serve to create a flow of air that is less disruptive to the normal flow of respiration. By changing the flow of air throughout the respiratory cycle the user experience may be viewed more positively and user acceptance may increase.

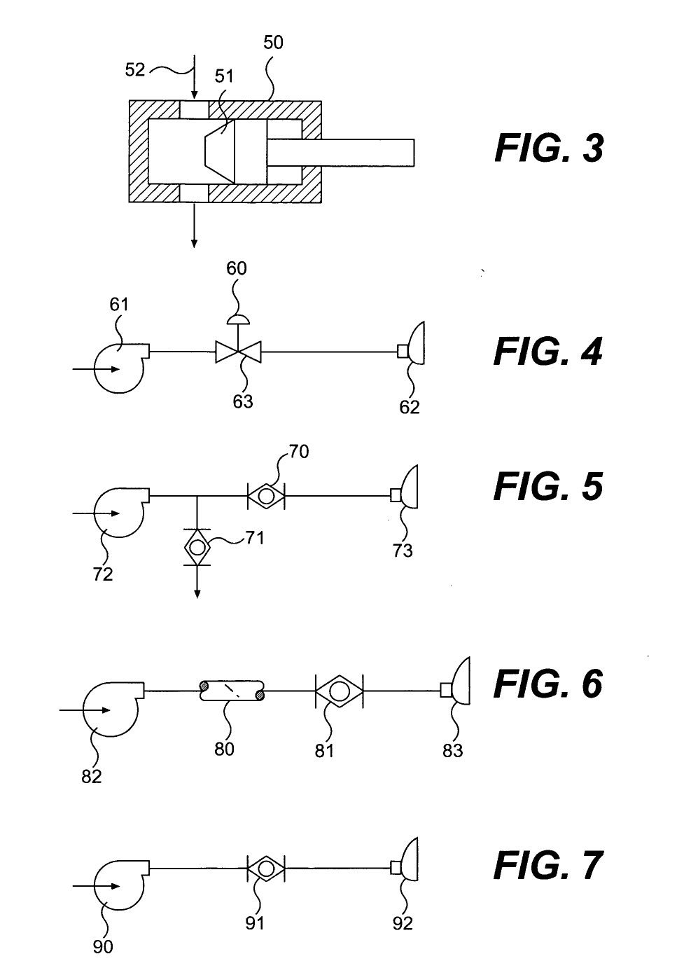

One problem that has been discovered in clinical studies is that the abrupt termination of a metered puff of air (any amount less than a full inspiratory volume of air) can arouse a patient. In order to address this potential issue it is preferred to taper the shutoff of puffs of air rather than abruptly shutting them off. A number of ways of solving this tapering problem are demonstrated in the schematic diagrams of FIGS. 3-7.

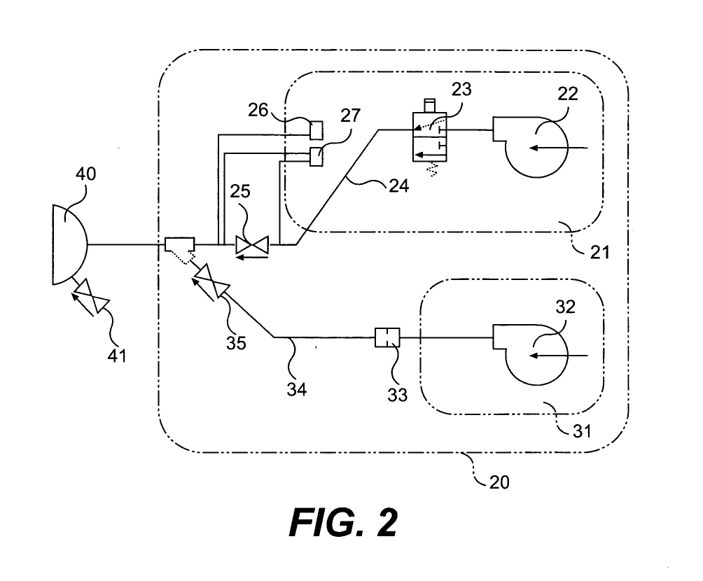

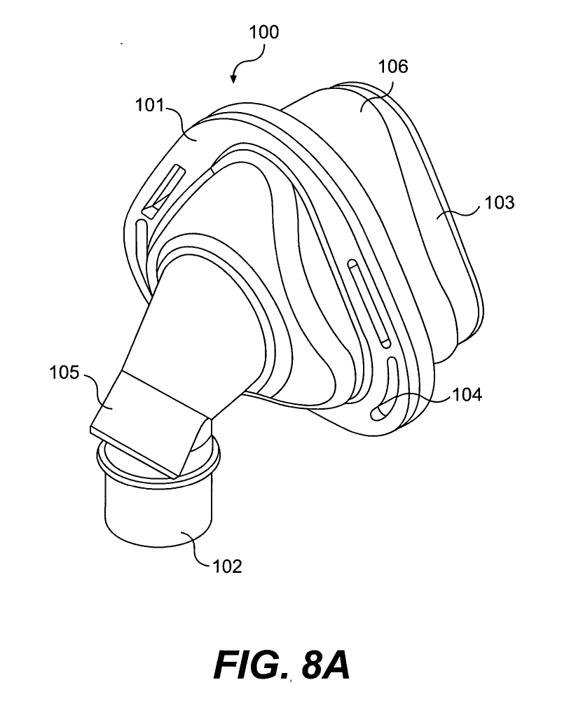

In FIG. 3, it is demonstrated that the valve construction itself could allow its shut off to be tapered. The valve 50 has a tapered spool 51. This construction could be effective in a solenoid driven spool valve 50 as shown. The air flow 52 is demonstrated by the arrows in FIG. 3. A further alternative is demonstrated in FIG. 4. It would be to use a variable speed valve actuator 60 that controls the speed at which the valve 63 closes. The valve 63 is placed between the blower 61 and the mask 62. An additional alternative (FIG. 5) would be to use two exhaust valves or ports 70 and 71. The second exhaust valve 71 would divert a portion of the air prior to a complete shut off of the puff from the blower 72 as delivered to the mask 73. Still further, as shown in FIG. 6, an in-line diverter such as a butterfly flap 80 can control the amount of air being delivered prior to complete puff shut off. As shown, the air from the blower 82 can be controlled by the butterfly flap 80 as well as the valve 81 before reaching the mask 83. A still further alternative, that is demonstrated in FIG. 2, provides a second level of air flow that is delivered both during and in the absence of a puff of air. This cushion of air is similar to or could be similar to the bias flow discussed herein. This cushion of air or bias flow could also be accomplished by engineering a valve that does not completely shut off. Finally, as demonstrated in FIG. 7, a variable speed blower 90 can ramp down the amount of air being delivered through the valve 91 prior to complete shut off of air flow to the mask 92. Other alternative constructions could be engineered by those of skill in the art to taper a reduction or complete shut off of air flow.

### EXAMPLE

A drawing displaying a working prototype of the present invention is set forth in FIG. 2. The system includes two air flow generator units 21 and 31 encased in a sound dampening suitcase 20. The two air flow generator units 21 and 31 serve different purposes. Air flow generator unit 21 generates the therapeutic puff described herein.

Air flow generator unit 31 generates a sub-therapeutic flow of air, referred to as a bias flow of air, for delivery to the mask 40. This bias flow of air from unit 31 can serve multiple purposes, but one leading purpose is to vent the mask 40 to prevent unwanted build up of condensation inside the mask 40. The unit 31 includes a blower 32. The air from the blower 32 is delivered by a d inch bore hose 34 to the mask 40 through check valve 35. A flow meter 33 monitors the specific flow of air from the unit 31.

The unit 21 creates the puff of the present invention. Blower 22 is connected by a 25 mm bore hose to the mask 40. The air leaves the blower 22 and passes through an on/off solenoid valve 23 and through check valve 25. A pressure transducer 26 and flow transducer 27 are used to detect triggering biological events in a patient. The transducers 26 and 27 sends signals to the solenoid valve 23 to actuate it open and closed. The mask 40 includes a check valve 41.

While the invention has been described with reference to specific embodiments thereof, it will be understood that numerous variations, modifications and additional embodiments are possible, and accordingly, all such variations, modifications, and embodiments are to be regarded as being within the spirit and scope of the invention.

1. A method of providing multi-level positive air pressure to the airway of a patient for breathing by the patient, the method comprising the steps of:
providing an air flow generator and a plurality of valves and hoses connected to and adapted to control and direct the flow of air from the air flow generator;
providing a mask that is connected to the air flow generator by the hoses, the mask adapted to deliver air to the airway of a patient;
actuating the plurality of valves to deliver multiple, different levels of positive air pressure to the airway of a patient.

2. A method of providing multi-level positive air pressure to the airway of a patient as described in claim 1, wherein the air flow generator comprises a plurality of blower apparatuses, and the plurality of valves and hoses are connected to the corresponding plurality of blower apparatuses.

3. A method of providing multi-level positive air pressure to the airway of a patient as described in claim 2, wherein the plurality of blower apparatuses each deliver different air flows.

4. A method of providing multi-level positive air pressure to the airway of a patient as described in claim 1, further comprising providing a plurality of physiological detectors adapted to detect biological triggering events; and wherein the detectors are connected to and actuate the valves.

5. A method of providing multi-level positive air pressure to the airway of a patient as described in claim 4, wherein the plurality of physiological detectors identify a corresponding plurality of different biological triggering events.

6. A method of providing multi-level positive air pressure to the airway of a patient as described in claim 1, wherein one of the positive air pressures constitutes a bias flow of air to the mask.

7. A method of providing multi-level positive air pressure to the airway of a patient as described in claim 1, further comprising providing a physiological detector adapted to detect a biological triggering event; and wherein the detector is connected to and actuates the valves.

8. An apparatus for providing multi-level positive air pressure to the airway of a patient for breathing by the patient, the apparatus comprising:
an air flow generator;
a plurality of hoses connected to the air flow generator and adapted to direct the flow of air from the air flow generator;
a corresponding plurality of valves connected to the plurality of hoses and adapted to control the flow of air from the air flow generator;
a mask that is connected to the air flow generator by the hoses, the mask adapted to deliver air to the airway of a patient.

9. An apparatus as described in claim 8, wherein the air flow generator comprises a plurality of blower apparatuses, and the plurality of valves and hoses are connected to the corresponding plurality of blower apparatuses.

10. An apparatus as described in claim 8, wherein the plurality of blower apparatuses each deliver different air flows.

11. An apparatus as described in claim 8, further comprising a plurality of physiological detectors adapted to detect biological triggering events, and wherein the detectors are connected to and actuate the valves.

12. An apparatus as described in claim 8, further comprising a physiological detector adapted to detect a biological triggering event, and wherein the detector is connected to and actuates the valves.

13. A method of providing multi-level positive air pressure to the airway of a patient for breathing by the patient, the method comprising the steps of:
providing an air flow generator, wherein the generator can provide multiple, different air flows;
providing a hose and a mask, the hose connected to the air flow generator and the mask to deliver air from the generator to the mask, the mask adapted to deliver the air to the airway of a patient; and
actuating the air flow generator to deliver multiple, different levels of positive air pressure to the airway of a patient.

14. A method of providing multi-level positive air pressure to the airway of a patient as described in claim 13, wherein one of the positive air pressures constitutes a bias flow of air to the mask.

15. A method of providing multi-level positive air pressure to the airway of a patient as described in claim 13, further comprising providing a physiological detector adapted to detect a biological triggering event; and wherein the detector is connected to and actuates the air flow generator.

16. A method of providing multi-level positive air pressure to the airway of a patient for breathing by the patient, the method comprising the steps of:
providing an air flow generator and a valve and hose connected to and adapted to control and direct the flow of air from the air flow generator;
wherein the valve is adapted to allow multiple, different levels of air to flow through it;
providing a mask that is connected to the air flow generator by the hose, the mask adapted to deliver air to the airway of a patient;
actuating the valve to deliver multiple, different levels of positive air pressure to the airway of a patient.

17. A method of providing multi-level positive air pressure to the airway of a patient as described in claim 16, wherein one of the positive air pressures constitutes a bias flow of air to the mask.

18. A method of providing multi-level positive air pressure to the airway of a patient as described in claim 16, further comprising providing a physiological detector adapted to detect a biological triggering event; and wherein the detector is connected to and actuates the valve.

19. A breathing mask for use in connection with a positive air flow source, the mask comprising:
a flexible interface adapted to fit around the nose and/or mouth of a patient, and
a rigid retainer adapted to fit securely around the flexible interface.

20. The breathing mask described in claim 19, wherein the rigid retainer is adapted to be removably fitted around the flexible interface.
